(19) United States
(12) Patent Application Publication
(10) Pub. No.: US 2014/0100472 A1
(43) Pub. Date: Apr. 10, 2014

(54) Systems and Methods for an Optical Nanoscale Array for Sensing and Recording of Electrically Excitable Cells

(71) Applicant: The Trustees of Columbia University in the City of New York, New York, NY (US)

(72) Inventors: Dirk R. Englund, New York, NY (US); Aurel A. Lazar, New York, NY (US); Chaitanya Rastogi, Paramus, NJ (US); Yevgeniy B. Slutskiy, Brooklyn, NY (US)

(73) Assignee: The Trustees of Columbia University in the City of New York, New York, NY (US)

(21) Appl. No.: 14/104,931

(22) Filed: Dec. 12, 2013

Related U.S. Application Data
(63) Continuation of application No. PCT/US2012/042255, filed on Jun. 13, 2012.
(60) Provisional application No. 61/496,388, filed on Jun. 13, 2011.

(51) Int. Cl. A61B 5/04; A61B 5/00

(52) U.S. Cl. CPC A61B 5/04001; A61B 5/7228
USPC 600/547

(57) ABSTRACT

Systems for untethered sensing and recording of activity of one or more electrically excitable cells in a target region includes at least one untethered probe. Each untethered probe includes at least one signal detector, configured to electrically couple to the target region, measure the activity of the one or more electrically excitable cells, and produce an electrical signal in response to the activity of the one or more electrically excitable cells, and at least one light source, electrically coupled to the at least one signal detector, to receive the electrical signal and emit a light signal representing the activity of the one or more electrically excitable cells. Methods for untethered sensing and recording of activity of one or more electrically excitable cells in a target region are also provided.

### CROSS-REFERENCE TO RELATED APPLICATIONS

This application claims priority to PCT/US12/042255, filed Jun. 13, 2012, which claims priority to U.S. Provisional Patent Application Ser. No. 61/496,388, filed on Jun. 13, 2011, the entirety of the disclosures of both of which are incorporated by reference herein.

### BACKGROUND

Extracellular recordings from one or more electrically excitable cells, for example neurons, of a subject can be performed with one or more glass or tungsten electrodes. The electrodes can be placed at or proximate to a target region of the subject to allow for in vivo extracellular recordings. However, performing recordings from a plurality of neurons in-vivo can be difficult at least in part due to the relatively small size of the target region containing neurons. Further, performing recording and clarifying the function of neuronal circuits associated with the neuronal recordings in-vivo in freely moving subjects can also be difficult.

### SUMMARY

Systems and methods for untethered sensing and recording of activity of electrically excitable cells are provided herein.

According to one aspect of the disclosed subject matter, a system for untethered sensing and recording of activity of one or more electrically excitable cells in a target region includes at least one untethered probe. Each untethered probe can include at least one signal detector, configured to electrically couple to the target region, measure the activity of the one or more electrically excitable cells, and produce an electrical signal in response to the activity of the one or more electrically excitable cells; and at least one light source, electrically coupled to the at least one signal detector, to receive the electrical signal and emit a light signal representing the activity of the one or more electrically excitable cells.

In some embodiments, the one or more electrically excitable cells can include one or more neurons. The at least one signal detector can include an electric field sensor. The system can further include at least one electrical contact to couple the at least one signal detector to the target region.

In some embodiments, the at least one light source can include a photonic cavity. The light source can include a semiconductor laser, and in some embodiments, the semiconductor laser can include a photonic crystal cavity.

In some embodiments, the at least one signal detector can be further configured to modulate at least one of an output intensity, an output wavelength and an output phase of the at least one light source in response to the activity. The light signal can be configured to encode a neuronal spike train.

In some embodiments, the system can further include an excitatory source to produce a pumping signal. The excitatory source can be optically coupled to the at least one light source, and the at least one light source can be further configured to emit the light signal in response to the electrical signal and the pumping signal. The system can further include an optical receiver, optically coupled to the at least one light source, configured to receive the light signal. The activity can include an electric field potential of the one or more electrically excitable cells, and the system can further include a processor, coupled to the optical receiver, configured to determine the electric field potential from the received light signal.

In some embodiments, the at least one untethered probe can include a plurality of untethered probes, and the at least one light source of each untethered probe can be configured to emit a corresponding one of a plurality of light signals, each light signal having a different wavelength or being emitted at a different time. The system can further include an optical demodulator/demultiplexor, optically coupled to the at least one light sources of the plurality of untethered probes, configured to record the light signals of more than one of the plurality of untethered probes substantially simultaneously.

According to another aspect of the disclosed subject matter, a method for untethered sensing and recording of activity of one or more electrically excitable cells in a target region includes measuring the activity of the one or more electrically excitable cells in the target region, producing an electrical signal in response to the activity of the one or more electrically excitable cells, and emitting a light signal representing the activity in response to the electrical signal.

In some embodiments, the method can further include modulating at least one of an intensity, a wavelength and a phase of the light signal in response to the activity. The method can further include encoding a spike train using the light signal.

In some embodiments, the method can further include pumping a light source with a pumping signal, and the light signal can be emitted by the at least one light source in response to the electrical signal and the pumping signal. The activity can include an electric field potential of the one or more electrically excitable cells, and the method can further include receiving the light signal and determining the electric field potential from the received light signal.

In another embodiment of the disclosed subject matter, a method for untethered sensing and recording of activity of one or more electrically excitable cells in a target region includes receiving a plurality of electrical signals in response to the activity of the one or more electrically excitable cells in the target region, and emitting a plurality of light signals, each light signal having a different wavelength or being emitted at a different time, and each light signal representing the activity in the target region in response to a corresponding one of the plurality of electrical signals. In some embodiments, the method can further include receiving more than one of the plurality of light signals substantially simultaneously.

### BRIEF DESCRIPTION OF THE DRAWINGS

FIGS. 1A-1D illustrate an exemplary system for neuronal sensing and recording according to the disclosed subject matter.

FIGS. 2A-2D illustrate an exemplary photonic crystal cavity of the system of FIGS. 1A-1D.

FIGS. 3A-3B illustrate exemplary laser nanoprobe structures of the system of FIGS. 1A-1D.

FIGS. 4A-4D illustrate exemplary embodiments of a semiconductor sensor and transmitter of the system of FIGS. 1A-1D.

FIGS. 5A-5B illustrate further aspects of neuronal sensing and recording using the system of FIGS. 1A-1D.

### DETAILED DESCRIPTION

One aspect of the disclosed subject matter relates to systems and methods for an optical nanoscale array for neuronal sensing and recording. The disclosed subject matter can be used, for example, for substantially simultaneous recording from one or more neurons and for recording of neuronal events in a freely moving subject.

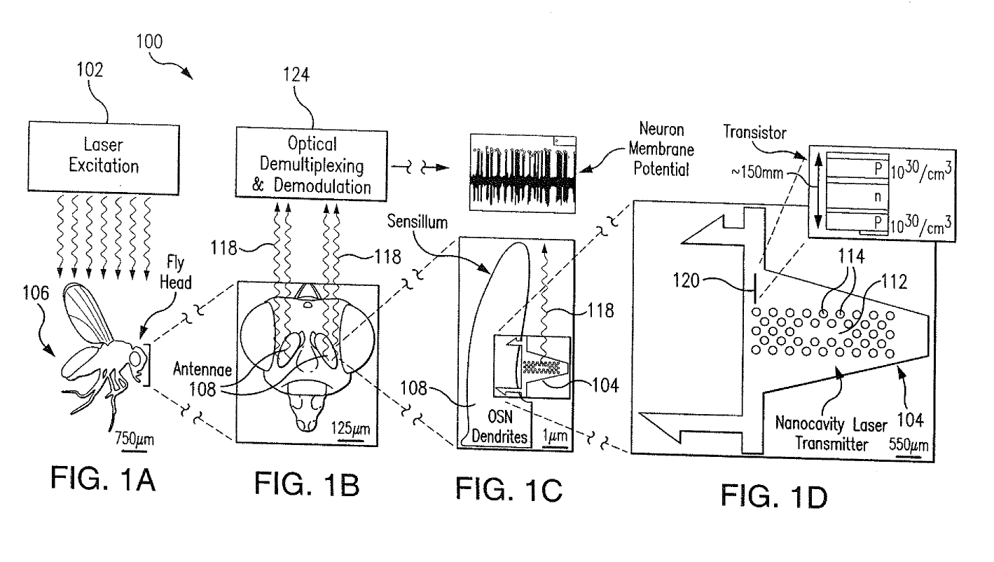

FIGS. 1A-1D illustrate an exemplary system 100 for neuronal sensing and recording according to the disclosed subject matter. As shown in FIG. 1A, an excitatory source 102, for example and embodied herein as a laser, can optically pump (i.e., provide power to) neuronal nanoprobes 104 attached to a subject 106. For example and as embodied herein, the subject 106 can be an insect, such as a fruit fly, or an animal or any other suitable subject providing neuronal activity to be recorded. In FIG. 1B, neuronal nanoprobes 104 can be attached to a target region 108 of the subject 106. For example and as embodied herein, the target region 108 can be fly antennae, and in some embodiments a sensillum of a fly antenna, or any other suitable region to optically transmit the activity of one or more neurons substantially simultaneously. In FIG. 1C, the neuronal nanoprobe 104 can be inserted into the target region 108 containing one or more neurons, and the target region 108 can, for example and as embodied herein, include dendrites of olfactory sensory neurons. Extracellular activity of the neurons can thus be encoded into a lightwave 118 by a nanocavity 112, which, for example and as embodied herein, can be a photonic crystal nanocavity. In FIG. 1D, the nanocavity 112 on a nanoprobe 104 can be configured as periodic arrangement of holes 114 acting as a reflective in-plane mirror.

In the nanoprobe 104, an electric field sensor with metal contacts can be coupled to a transducer module to modulate one or more of the laser output intensity, the center wavelength and the output phase. A number of different geometries and materials for the electric field sensor can be utilized to sense fields with a suitable signal-to-noise ratio. As described further below, the modulator can include one or more of an integrated bipolar transistor 120, a quantum confined Stark effect modulator, and an electrostatically actuated opto-mechanical laser cavity 122. Several nanoprobes 104 can be attached to the target region 108 to demonstrate neuronal sensing and recording from a population of neurons in a subject.

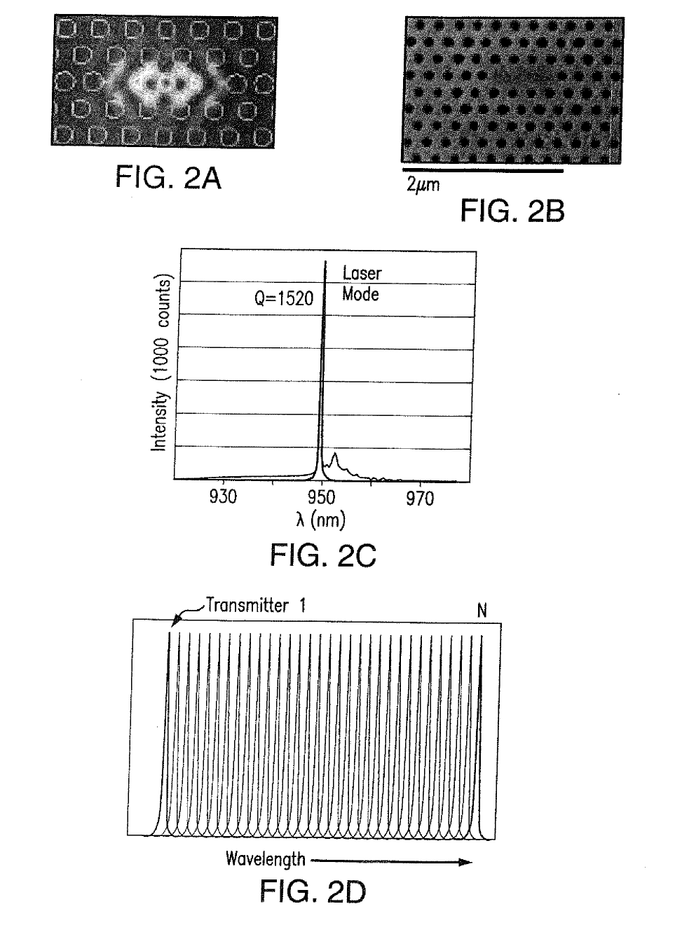

The nanocavity 112, for example and as embodied herein, can be an optically-pumped semiconductor laser and can include a gain medium enclosed in an optical cavity. Carriers in the gain medium can be excited by an external light source and drive cavity modes through stimulated emission. Several cavity geometries can be utilized, and in some embodiments, an optical cavity defined in a planar photonic crystal (PC) cavity can be used. FIGS. 2A-2D illustrate an exemplary photonic crystal cavity. FIG. 2A is a diagram illustrating a cavity mode. FIG. 2B is a diagram illustrating a GaAs PC with an InAs quantum dot (QD). FIG. 2C is a diagram illustrating a single-cavity spectrum. FIG. 2D is a diagram illustrating dense wavelength division multiplexing (DWDM) from several probe sites.

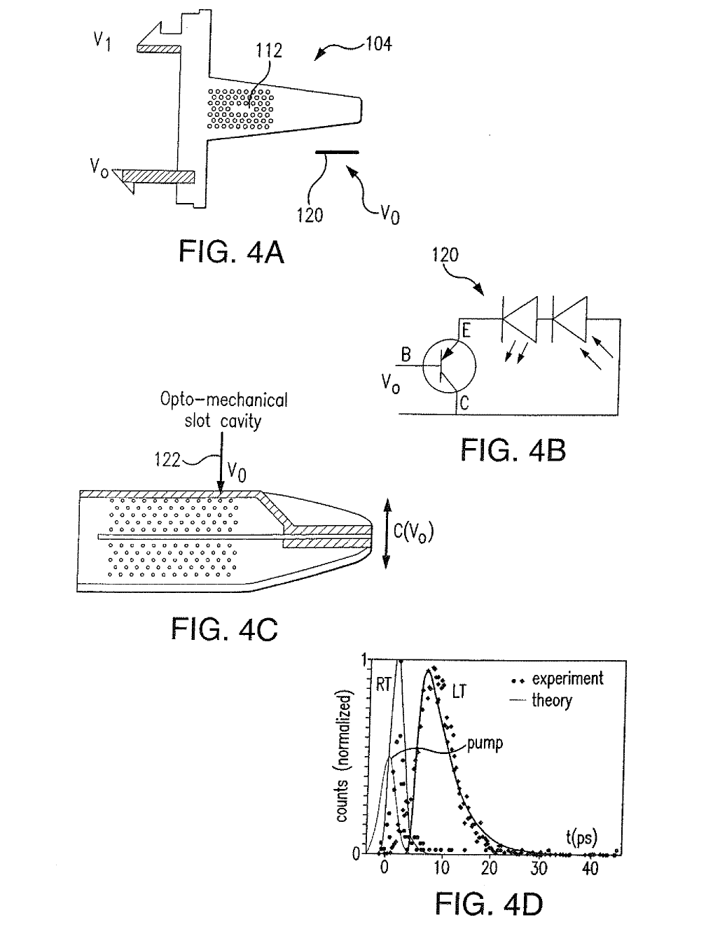

The PC confinement can be sufficiently high to confine light below the so-called diffraction limit. A PC membrane, for example and as embodied herein, can include a 130-nm thick GaAs membrane patterned with a periodic arrangement of holes and can be utilized as a highly reflective in-plane mirror, as illustrated in FIGS. 2A-2B. The gain medium, for example a central layer of InAs quantum dots (QDs) or any other suitable gain medium, can provide gain at a wavelength that can be tuned based on various factors, such as the absorption of tissue of the subject. For example, with the PC membrane embodied herein as a GaAs membrane, a suitable range of the wavelength can be between about 950-1300 nm. The PC cavity can allow relatively high light confinement to less than a volume of (λ/n)³, where λ/n can be considered as the laser wavelength, where n represents a refractive index of the material (for example, for the GaAs material, n can be approximately 3.5). The PC laser can be suitable as an optical transmitter of cell signals, for example because PC laser can be operated in liquid. Further, the laser structure can be relatively small (i.e., on the order of several cubic microns in volume) compared to other narrow-linewidth semiconductor lasers, and the weight can be, for example on the order of 1 pg, which can be relatively light compared to a target region 108 of a subject 106, which, for example and as embodied herein, can be about 10 pg for the small basiconic sensillum and 30 pg for the large basiconic sensillum of a fruit fly. A PC nanocavity laser can also have relatively high efficiency and can have a relatively low lasing threshold compared to other laser geometries, due at least in part to the Purcell effect of the laser. Further, the laser geometry can be relatively flat and open, which can present sufficient surface for efficient optical pumping. As a result, a relatively low external pump intensity, which, for example, can be suitable for insect or animal studies, can be used. Additionally, a relatively small emission linewidth (i.e., as low as about 0.01 nm) and single-mode operation can be suitable for dense wavelength division multiplexing (DWDM) to track multiple channels, for example as illustrated in FIGS. 2C-2D. Telecom wavelength operation can be configured, which can allow a range of advanced and relatively low-cost opto-electronics equipment to be utilized. Additionally or alternatively, time division multiplexing (TDM) can be performed to track multiple channels, whereby a plurality of nanoprobes 112 can operate at the same frequency. As such, the output of the nanoprobes 112 can be detected sequentially in time, for example using spatial scanning hardware, such as a mirror galvanometer. The PC laser can also have ps-gain modulated operation (for example, as shown in FIG. 4D), which can provide suitable modulating speed for the system of the disclosed subject matter.

As described above, an array of untethered nanoprobes 104 that can be attached to a relatively small target region 108, such a sensillum of a fruit fly, can be difficult to construct due to the size of the target region 108. Fabrication guidelines for the nanoprobes 104 can be determined based on the size of and the additional weight placed on the target region 108. Furthermore, the nanoprobes 104 can be configured to operate at certain wavelengths so as to not interfere with the light spectrum perceived by a subject 106. Additionally, nanoprobes 104 can be configured to have relatively low heat dissipation to avoid interference with thermal receptors of a subject 106.

For example and without limitation, a nanocavity 112, embodied herein as a PC laser in a GaAs membrane, can include one or more layers of InAs quantum dots, which can emit light having a wavelength within a range between about 900-980 nm. The laser nanoprobe structures can be fabricated, for example and without limitation, using electron beam lithography in polymethyl methacrylate (PMMA), followed by a plasma-etch mask transfer and a wet-etch removal of a sacrificial layer beneath the membrane.

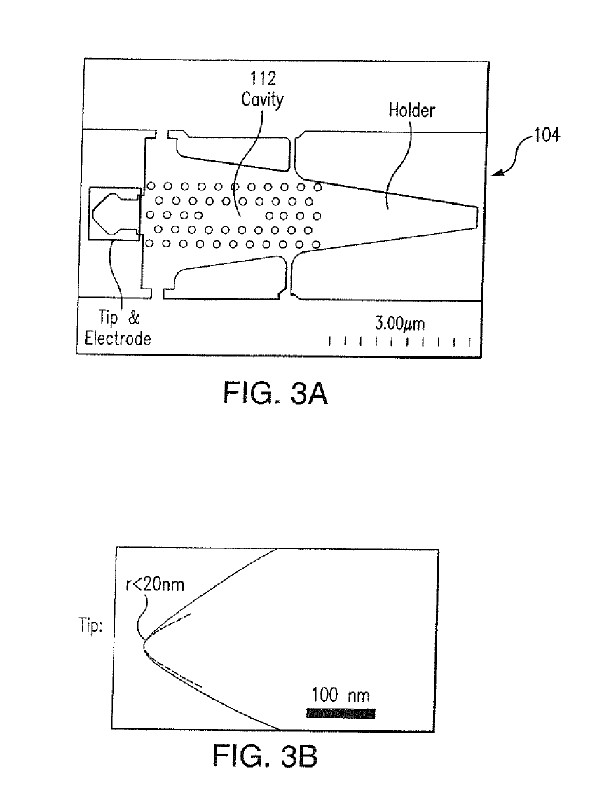

To reduce nonradiative (NR) surface recombination, for example and without limitation, the laser nanoprobe structures can be passivated and conformally capped with a cyto-compatible material, such as aluminum oxide. The laser nanoprobe structures can be pumped optically with about 3-ps short pulses at about an 80 MHz repetition rate, or using a continuous-wave pump, at a wavelength centered at about 750 nm. At room temperature, the photoluminescence of the In_{0.2}Ga_{0.8}As quantum wells can peak at about 980 nm. Immersing the photonic crystal membrane in water or saline can improve heat dissipation by up to about 20×, based on measurements of the maximum pump power before the structure is damaged. Exemplary laser nanoprobe structures are illustrated in FIGS. 3A-3B. FIG. 3A is a scanning electron micrograph of the laser nanoprobe. FIG. 3B is an image showing that the tip radius can be less than about 20 nm.

The laser nanocavity 112 can be optically pumped using an excitatory source 102, for example and as embodied herein an external laser, which can emit a pulsed laser beam, for example at a wavelength of about 830 nm, which can be invisible to the subject 106. The center wavelength, pulse frequency, and duty cycle can be selected for improved pump efficiency and signal read-out. The pulse energy to reach lasing threshold can be 10⁻¹² J or less. A pulse frequency of 1 MHz, which can be a sufficient sampling rate of the cell potential, can provide an average power of about 1 μW, which can operate without significantly changing the surface temperature of the animal. For purpose of comparison, two-photon microscopy is generally performed at on the order of tens of mWs.

FIGS. 4A-4D illustrate exemplary embodiments of a semiconductor sensor and transmitter according to the disclosed subject matter. Several techniques can be utilized to modulate the output of the nanocavity 112 by the cell electric field potential. For example, in some embodiments, the output of the nanocavity can be modulated using one or more lasers. In one embodiment, for example as shown in FIGS. 4A-4B and without limitation, a bipolar transistor 120 can be integrated vertically, or laterally (for example by using ion implantation), into a GaAs laser membrane to modulate the optically pumped laser intensity. Thus, the gain medium in the laser probe can be optically pumped and emit vertically. In another embodiment, as shown in FIG. 4C, the optically-pumped laser intensity can be modulated using an opto-mechanical planar PC cavity 122, which can be actuated electrostatically using a built-in capacitor to shift the cavity center wavelength. Using a nanocavity 112 with a linewidth of about 0.1 nm can provide a shift greater than 5 cavity linewidths/10 mV, which can be sufficient for the system of the disclosed subject matter. Thus, the signal can be transmitted using cell-voltage controlled emission of an optically pumped laser diode. In some embodiments, the optically-pumped laser intensity can be modulated using a cavity. For example, in another embodiment, the cavity can include a quantum confined Stark effect (QCSE) modulator disposed across an InAs quantum well (QW) or quantum dot (QD) layer. An exemplary QCSE modulator is shown and described, for example and without limitation, in Fast Electrical Control of a Quantum Dot Strongly Coupled to a Photonic-Crystal Cavity by Faraon et al., Physical Review Letters 104 (4): 047402-1-4 (Jan. 29, 2010), the disclosure of which is incorporated by reference herein in its entirety. The QW can also operate as the laser gain medium, after passivation and encapsulation. In another embodiment, the cavity can include an opto-mechanical planar PC cavity 122 to modulate the laser intensity as described above. However, where sensing and recording is performed using a relatively viscous medium, the opto-mechanical planar PC cavity 122 can be less effective than other techniques due at least in part to restricted mechanical movement in the viscous medium. FIG. 4D illustrates the rapid laser response (i.e., after the pump pulse) for room temperature (RT) operation of the nanocavity 112.

### EXAMPLE

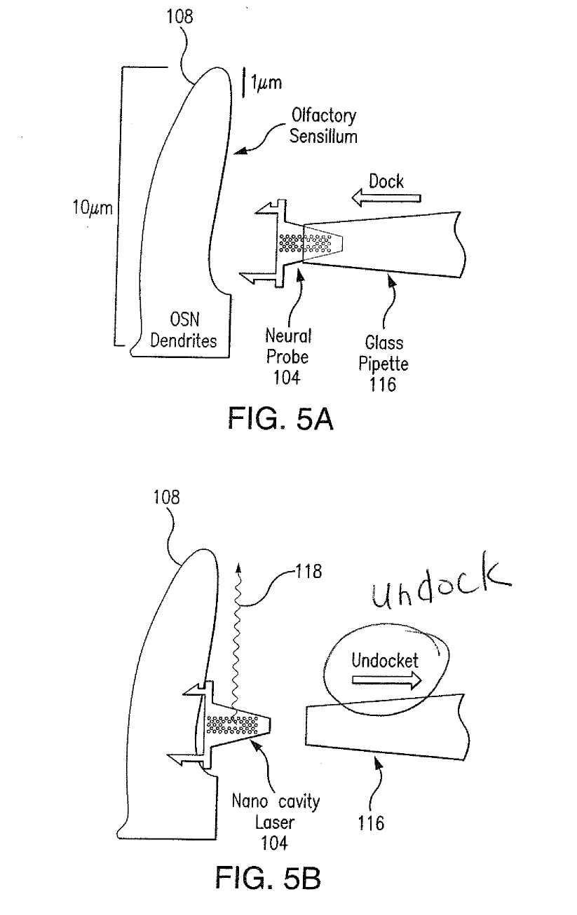

According to the disclosed subject matter, recording of spike trains can be performed untethered, as illustrated in FIGS. 5A-5B. For example and as embodied herein, to record responses of olfactory sensory neurons, a glass pipette 116 can be used to pick up a neuronal probe 104 and attach it to the target region 108, embodied herein as an olfactory sensillum (as shown in FIG. 5A). For example, and as embodied herein, the tip of the glass pipette 116 can be filled with water, and the pipette itself can be mounted on a motorized micro-manipulator. Low/high air pressure within the glass pipette 116 can be used to facilitate docking/undocking of the neuronal probe 104 to/from the glass pipette (as shown in FIGS. 5A-5B). Once inside the target region 108, the probe 104 can contact a conductive lymph surrounding the dendritic tree of a neuron and measure the extracellular activity of that neuron. That is, an action potential generated by a neuron can propagate back through lymph and dendrites and create a detectable potential gradient within the target region 108. A potential drop between two harpoons of the probe 104 can then be used to modulate a light wave emitted by a nanocavity. With each probe 104 operating at a different wavelength, an optical demodulator/demultiplexor 124 can be used to record response of several neurons substantially simultaneously, for example as illustrated in FIG. 1B. The optical demodulator/demultiplexor 124 can be implemented using any suitable device for demodulating and demultiplexing an optical signal.

An application of the disclosed subject matter includes identifying dendritic processing in a neuronal circuit. One method for identifying dendritic processing in a class of phenomenological neuronal circuit models is described in U.S. patent application Ser. No. 13/249,692, filed Sep. 30, 2011, the entirety of the disclosure of which is explicitly incorporated by reference herein. The method provides that linear processing can take place in a dendritic tree, and the resulting aggregate dendritic current can be encoded by a spiking neuron. An estimate of the dendritic processing (i.e., a dendritic processing filter) can be based on a single spike train corresponding to a single stimulus instance.

While the disclosed subject matter is described herein in terms of certain exemplary embodiments, those skilled in the art will recognize that various modifications and improvements can be made to the disclosed subject matter without departing from the scope thereof. For example, while the exemplary embodiments herein describe sensing the electrical activity associated with sensory neurons of fruit flies, the systems and methods disclosed herein can be suitable for a variety of other applications. That is, systems and methods disclosed herein can be used for neuronal sensing and recording performed on any suitable animal, including insects and vertebrates. Further, the systems and methods described herein can be used to sense and record local neurons and projection neurons in the olfactory lobe of the fruit fly, as well in early vision. For example, systems and methods according to the disclosed subject matter can be adapted for monitoring the simultaneous activity of tangential cells in the lobula plate. As such, the systems and methods according to the disclosed subject matter can be utilized for a variety of neuronal sensing and recording applications.

The foregoing merely illustrates the principles of the disclosed subject matter. Various modifications and alterations to the described embodiments will be apparent to those skilled in the art in view of the teachings herein. It will be appreciated that those skilled in the art will be able to devise numerous modifications which, although not explicitly described herein, embody its principles and are thus within its spirit and scope.

We claim:

1. A system for untethered sensing and recording of activity of one or more electrically excitable cells in a target region, comprising:
at least one untethered probe comprising:
at least one signal detector, configured to electrically couple to the target region, measure the activity of the one or more electrically excitable cells, and produce an electrical signal in response to the activity of the one or more electrically excitable cells; and
at least one light source, electrically coupled to the at least one signal detector, to receive the electrical signal and emit a light signal representing the activity of the one or more electrically excitable cells.

2. The system of claim 1, wherein the one or more electrically excitable cells comprises one or more neurons.

3. The system of claim 1, wherein the at least one signal detector comprises an electric field sensor.

4. The system of claim 1, further comprising at least one electrical contact to couple the at least one signal detector to the target region.

5. The system of claim 1, wherein the at least one light source comprises a photonic cavity.

6. The system of claim 1, wherein the at least one light source comprises a semiconductor laser.

7. The system of claim 6, wherein the semiconductor laser comprises a photonic crystal cavity.

8. The system of claim 1, wherein the at least one signal detector is further configured to modulate at least one of an output intensity, an output wavelength or an output phase of the at least one light source in response to the activity.

9. The system of claim 1, wherein the light signal is configured to encode a neuronal spike train.

10. The system of claim 1, further comprising an excitatory source to produce a pumping signal, the excitatory source optically coupled to the at least one light source, the at least one light source further configured to emit the light signal in response to the electrical signal and the pumping signal.

11. The system of claim 1, further comprising an optical receiver, optically coupled to the at least one light source, configured to receive the light signal.

12. The system of claim 11, wherein the activity comprises an electric field potential of the one or more electrically excitable cells, the system further comprising a processor, coupled to the optical receiver, configured to determine the electric field potential from the received light signal.

13. The system of claim 1, wherein the at least one untethered probe comprises a plurality of untethered probes, the at least one light source of each untethered probe being configured to emit a corresponding one of a plurality of light signals, each light signal having a different wavelength or being emitted at a different time.

14. The system of claim 13, further comprising an optical demodulator/demultiplexor, optically coupled to the at least one light sources of the plurality of untethered probes, configured to record the light signals of more than one of the plurality of untethered probes substantially simultaneously.

15. A method for untethered sensing and recording of activity of one or more electrically excitable cells in a target region, comprising:
measuring the activity of the one or more electrically excitable cells in the target region;
producing an electrical signal in response to the activity of the one or more electrically excitable cells; and
emitting a light signal representing the activity in response to the electrical signal.

16. The method of claim 15, further comprising modulating at least one of an intensity, a wavelength and a phase of the light signal in response to the activity.

17. The method of claim 15, further comprising encoding a neuronal spike train using the light signal.

18. The method of claim 15, further comprising pumping a light source with a pumping signal, wherein the light signal is emitted by the at least one light source in response to the electrical signal and the pumping signal.

19. The method of claim 15, wherein the activity comprises an electric field potential of the one or more electrically excitable cells, the method further comprising receiving the light signal and determining the electric field potential from the received light signal.

20. The method of claim 15, wherein the one or more electrically excitable cells comprises one or more neurons.

21. A method for untethered sensing and recording of activity of one or more electrically excitable cells in a target region, comprising:
receiving a plurality of electrical signals in response to the activity of the one or more electrically excitable cells in the target region; and
emitting a plurality of light signals, each light signal having a different wavelength or being emitted at a different time, and each light signal representing the activity in the target region in response to a corresponding one of the plurality of electrical signals.

22. The method of claim 21, further comprising receiving more than one of the plurality of light signals substantially simultaneously.
